SEQUENCE LISTING (10 sequences)

SEQ ID NO: 1 - DNA, 1305 bp, Magnetospirillum magneticum
atggaactgcatcatcccgaactgacctatgccgccatcgtcgccctggccgccgtgctgtgcggcgggatgatgacgcgcctgaagcagccggccgtcgtcggctacatcctggcgggggtggtgctgggacccagcggcttcgggctggtgagcaaccgcgacgccgtggccaccctggccgagttcggcgtgctgatgctgctgttcgtcatcggcatgaagctggacatcatccgctttctcgaagtgtggaagacggccatcttcaccacggttctgcagatcgccggcagcgtgggcacggccctgctgctgcgtcacggcctgggctggagcctggggctggcggtggtgctgggctgtgccgtggcggtgtcgtccaccgccgtagtgatcaaggtgctggaatcctcggacgagctggacacgccggtcggccgcaccacccttggcatcctgatcgcccaggacatggcggtggtgcccatgatgctggtgctggaatccttcgagaccaaggcgctgctgcccgccgacatggcccgggtggtgctgtcggtgctgttcctggtgctgctgttctggtggctgtccaagcgccgcatcgacctgccgatcaccgcccggctttcccgcgattctgaccttgccaccctgtcgaccctggcctggtgtttcggcaccgccgccatctccggcgtgctggacttgtcgccggcctatggcgccttcctgggcggcgtggtgctgggcaattccgcccagcgcgacatgctgttgaagcgtgcccagcccatcggcagcgtgctgctgatggtgttcttcctgtccatcgggctgctgctcgacttcaagttcatctggaagaatctgggcaccgttctcaccctgctggccatggtgaccctgttcaagacggcgctgaacgtcacggcgctgcgcctggcgcggcaggactggcccagcgccttcctggccggcgtggccctggcccagatcggcgagttctcgttcctgctggccgagaccggcaaggcggtcaagctgatcagcgcccaggagaccaagctggtggtggcggtcaccgtgctgtccctggtgctgtcgccgttctggctgttcaccatgcggcgcatgcaccgggtggcggcggtgcatgtccattcgttccgcgatctggtcacgcggctgtatggcgacgaggcccgcgctttcgcccgcaccgcgcggcgggcccgtgtgctggtgcggcgtggttcctggagggatgaccccaatgccggacctggctctggaatttga

SEQ ID NO: 2 - protein, 434 aa, Magnetospirillum magneticum
MELHHPELTYAAIVALAAVLCGGMMTRLKQPAVVGYILAGVVLGPSGFGLVSNRDAVATLAEFGVLMLLFVIGMKLDIIRFLEVWKTAIFTTVLQIAGSVGTALLLRHGLGWSLGLAVVLGCAVAVSSTAVVIKVLESSDELDTPVGRTTLGILIAQDMAVVPMMLVLESFETKALLPADMARVVLSVLFLVLLFWWLSKRRIDLPITARLSRDSDLATLSTLAWCFGTAAISGVLDLSPAYGAFLGGVVLGNSAQRDMLLKRAQPIGSVLLMVFFLSIGLLLDFKFIWKNLGTVLTLLAMVTLFKTALNVTALRLARQDWPSAFLAGVALAQIGEFSFLLAETGKAVKLISAQETKLVVAVTVLSLVLSPFWLFTMRRMHRVAAVHVHSFRDLVTRLYGDEARAFARTARRARVLVRRGSWRDDPNAGPGSGI

SEQ ID NO: 3 - DNA, 1374 bp, Artificial Sequence
  MagA sequence codon-optimized for Chlamydomonas
ctatctagaggatccccgggtggtctcttgaccatggagctgcaccaccccgagctgacctacgccgccatcgtggccctggccgccgtgctgtgcggcggcatgatgacccgcctgaagcagcccgccgtggtgggctacatcctggccggcgtggtgctgggccccagcggcttcggcctggtgagcaaccgcgacgccgtggccaccctggccgagttcggcgtgctgatgctgctgttcgtgatcggcatgaagctggacatcatccgcttcctggaggtgtggaagaccgccatcttcaccaccgtgctgcagatcgccggcagcgtgggcaccgccctgctgctgcgccacggcctgggctggagcctgggcctggccgtggtgctgggctgcgccgtggccgtgagcagcaccgccgtggtgatcaaggtgctggagagcagcgacgagctggacacccccgtgggccgcaccaccctgggcatcctgatcgcccaggacatggccgtggtgcccatgatgctggtgctggagagcttcgagaccaaggccctgctgcccgccgacatggcccgcgtggtgctgagcgtgctgttcctggtgctgctgttctggtggctgagcaagcgccgcatcgacctgcccatcaccgcccgcctgagccgcgacagcgacctggccaccctgagcaccctggcctggtgcttcggcaccgccgccatcagcggcgtgctggacctgagccccgcctacggcgccttcctgggcggcgtggtgctgggcaacagcgcccagcgcgacatgctgctgaagcgcgcccagcccatcggcagcgtgctgctgatggtgttcttcctgagcatcggcctgctgctggacttcaagttcatctggaagaacctgggcaccgtgctgaccctgctggcgatggtgaccctgttcaagaccgccctgaacgtgaccgccctgcgcctggcccgccaggactggcccagcgccttcctggccggcgtggccctggcccagatcggcgagttcagcttcctgctggccgagaccggcaaggccgtgaagctgatcagcgcccaggagaccaagctggtggtggccgtgaccgtgctgagcctggtgctgagccccttctggctgttcaccatgcgccgcatgcaccgcgtggccgccgtgcacgtgcacagcttccgcgacctggtgacccgcctgtacggcgacgaggcccgcgccttcgcccgcaccgcccgccgcgcccgcgtgctggtgcgccgcggcagctggcgcgacgaccccaacgccggccccggcagcggcatctaagctagcgtgtgaattggtgacccgagctcgaatttc

SEQ ID NO: 4 - DNA, 402 bp, Magnetospirillum magneticum
atgggcgagatggagcgcgagggcgccgccgccaaggccggggctgccaagacgggcgccgccaagaccggaaccgtcgccaagaccggcatcgccgccaagacgggtgttgccaccgccgttgccgctccggcggctcctgccaatgttgccgccgcccagggcgccgggaccaaggtcgcccttggcgcgggcaaggccgccgccggtgccaaggtcgtcggtggaaccatctggaccggtaaggggctgggcctcggtctgggtctcggtctgggcgcgtgggggccgatcattctcggcgttgttggcgccggggcggtttacgcrtatatgaagagccgtgatatcgaatcggcgcagagcgacgaggaagtcgaactgcgcgacgcgctggcctga

SEQ ID NO: 5 - DNA, 474 bp, Magnetospirillum magneticum
gtgccagctcagatcgccaacggagttatttgccccccaggggccccggccggaaccaaggccgccgccgccatgggcgagatggagcgcgagggcgccgccgccaaggccggggctgccaagacgggcgccgccaagaccggaaccgtcgccaagaccggcatcgccgccaagacgggtgttgccaccgccgttgccgctccggcggctcctgccaatgttgccgccgcccagggcgccgggaccaaggtcgcccttggcgcgggcaaggccgccgccggtgccaaggtcgtcggtggaaccatctggaccggtaaggggctgggcctcggtctgggtctcggtctgggcgcgtgggggccgatcattctcggcgttgttggcgccggggcggtttacgcgtatatgaagagccgtgatatcgaatcggcgcagagcgacgaggaagtcgaactgcgcgacgcgctggcctga

SEQ ID NO: 6 - protein, 133 aa, Magnetospirillum magneticum
MGEMEREGAAAKAGAAKTGAAKTGTVAKTGIAAKTGVATAVAAPAAPANVAAAQGAGTKVALGAGKAAAGAKVVGGTIWTGKGLGLGLGLGLGAWGPIILGVVGAGAVYAYMKSRDIESAQSDEEVELRDALA

SEQ ID NO: 7 - protein, 157 aa, Magnetospirillum magneticum
MPAQIANGVICPPGAPAGTKAAAAMGEMEREGAAAKAGAAKTGAAKTGTVAKTGIAAKTGVATAVAAPAAPANVAAAQGAGTKVALGAGKAAAGAKVVGGTIWTGKGLGLGLGLGLGAWGPIILGVVGAGAVYAYMKSRDIESAQSDEEVELRDALA

SEQ ID NO: 8 - DNA, 399 bp, Artificial Sequence
  Mms6 sequence codon-optimized for Chlamydomonas
atgggcgagatggagcgcgagggcgccgccgccaaggccggcgccgccaagaccggcgccgccaagaccggcaccgtggccaagaccggcatcgccgccaagaccggcgtggccaccgccgtggccgcccccgccgcccccgccaacgtggccgccgcccagggcgccggcaccaaggtggccctgggcgccggcaaggccgccgccggcgccaaggtggtgggcggcaccatctggaccggcaagggcctgggcctgggcctgggcctgggcctgggcgcctggggccccatcatcctgggcgtggtgggcgccggcgccgtgtacgcctacatgaagagccgcgacatcgagagcgcccagagcgacgaggaggtggagctgcgcgacgccctggcc

SEQ ID NO: 9 - DNA, 3974 bp, Chlamydomonas reinhardtii
acattctctgttcgcgacaggacaacgtccggcgagaacgcagatcaagtcggtcgggccggtctacaacaacggtcgcgacagcacttcttcgccgccccgtccgcctacaacaacggcagccgagagagccatcttaagtgcaaatcagtttactgctggtgctgtcaagatggggcccactccaagctgggctgcgcgcggacgcgctgcggtggcgctcagtgcgtccctgttctttcagctcctggtcttggcgggctgtgcaacattcgcattcttcatgtttgtgacgcccacccgctggtacctcgtccgcgaagccgagttcatagcctggcgcaactccctcatcctgccctacaccttcatcaacggcaccaccggccgcaccacccctctgctgagtgggaccataagctggctggttatctggcaatggaccacaaccctggcggcggcgctgggtgccgcgggcctgttgtgggtggcctccctcaactccgcctcttcccccactcccaataagagttcgtcttccaagcgcccgcaggcatgctggtccctgggacgcgcaattaaaagggccctcacccgtgcccgtcgcgtgctgcaccacccgctgcctccccgtggcatgtggcgcacgctgtttggccccgggggcctgagcgtcctggacatgttcttgatcctgttctggtttgcgatccacgccatgtggatgtacgagatgaccatgcgtgtcttggacaaccgccgtgccaacccgccgccgccccgaccccgcagccctcctccagcctccgccgcccggccacttcccgccgcatccaccagctcctccaccaacgcctccgcctccagcggcaccgcgtcctctcccaggcccgccgcccgtctcctgctaggcgccacccacagccgcaccctcctccagtcccagtcccagtcccagtccgcctacccgccgccgcccaagtccgccaacagcaccaaggccgccgcgcccccgccgccaaccgtgcggaggctgaagcccctgcccgaagtcgtacaggacagtgtcgccaagtactctggctgggtgggccgcctggacctgctgctgctgttcttccctctgccgcgctgcaacttcctgcactggctgctggggagcgacttccccaccttggtcaagtaccacaggtggttgggccacggcacgatgctgatcaactccatccacggcatcgtatacatgggcatgtgggccaacgccggcaccctgacgaccatgctgaattggggcatgggcgccggcgtcaatcgtcttgctggcctcatctcgctgtgcggcggctggctcctttggatcacctgcatccccatcattcgcaggcgcttcttcaacgttttctacatcagccacatcctcggcgcgatcatatttcttctgttcggcttcatgcaccgcaaggacgtggccacctgggtcatgccgggcatcttcctgtaccttctcgatgtggtgctgcggaccatccagcagtgcttcaacgcgacgcgcgtgtctctcgttgcatctcctgccgacactcctgcaagcatcaagcgcgccgtcatgtccatgccccagggcggcagcggcggtgtggctgcgagcctgtcccgtgacggcggcgtgctgaccctcaccatcccctgcgaccagtccatgacctggtgtggcggcgacatcgtgttcctgaacctgccctgtgtcagctggtggcagtggcaccccttcaccatcgccaacacctccgctacctttgaccaccccgccgccgccacaccctcgcccggcgccgagccccacttcgccaaggactccagcatgtcctccaccagcgcctcatcgggtgccgcggctgccgccgcctccggtgcacgtgtgcgctacatgcaggtgcacatcaagaagtacaatgcctggacccggagcctcatcaaacagctggaagcttccggcggcgcctcctccctggccctgtacgtcagcggcccctaccatccgcccaccctcaccggctccaacagctacagccgccgcctggtgaacggcttccggcgccacgtcttcattgcgggcggcattggggtcacacccgccctgggaatgatccaggagctgattgccgcacgccgcaacaccaacaccaacaacaacaccaacaacaccaacaccggcaccaatgacgacaaggcccacggccgcgtgacgttcgtgtgggtgagccgcagcgctgacgagctggaggcggtgctaccggccgaggtgctggaggaggccaaccggggcaaggacggttggcttgacatgcagctctacatcaccggcagtagcaacaacagcaacagcaaggccgccgccgcctcggcaatcacggtggacgttgccgcggacgcaaacaccgccgccggagccgccgacgcctcggctgctgccgctgccgaggccaagctcgccagcctcaccccggcgttctctcgctcctatgcgaccttcagccacgctctgtcaggcctcagcggccttcgccgcagtggcagcaacagcagcagcggcgacattggcagtggcctggccggcggcttctttggctttcgctccgtgtgggacccggtggccgcggttgtcttcctgcccggatacctgctggtgcttgcggccggtgccgtgcggtgggcggcgtttggagccgtcaaggccaggcctctggctcacccctacatcgcgggtgggccgctgatgtgggcggtggctgtggtgctgtgcttcgcgggcggcttcggcggactcatcaccgcacagtcgtacgacggttacgccgcccgtacggtggcggtgcggtcggactactcgtatgtgggcatgctgcagtttgcggcgctgggtgtgggcgcggtgctgggacccgcgctgctggccctggtgatgcacgccatgtgcaggcttctcagcctgaagaggcgcaccgccaacgtaaccgaaatggagtcacagccttccgcctcaggctccgacgcctcgcccgctgactgccgcatcggcgtcacggtcacagctggcaccaacgccgccaccggtgctgcctccaaggctctggccgcgccgccgccggctcacgtgcccaccgccgccgcctacatcagcaccggacgccccgacctcaagaccatcctcacgcacgccgccgcgccctacgccccggacggtgaggcggttgaggatgaccaggccgctgctgtgggcgttttcgtggccgggccggcccctctggtggcggctgtggcggacacgtgcgctgctgtgaacggtgtgtgggctgcgctgtttggcggctgctgcggtggcggtgtgggtggccgtgcttacctggagctgcacgcgctgacgcatgagctgtagagtgcatgcgggtacatgacgtaggctgggtgctggcggagtggtggtggagcgtgctggagcactcactttgcgggcaatcagtgacgaacggtgacggtgcgtggtggcgatggtgatgggtgctatatataagtaaggcgagcggctgtttatttatcgcactgctgccgtcggacggagcgaggaggcaacaggttgtggcccatagaagtcatgagtgtgcgcaccagcagtgaagccttccatttagcgcaaccttgcttgcttgcatgctatctacacatacacatgcttggcggcggttaggtacagtgtcccgtccctacctccgttgtgggcactgtgcgtgtttgctgctggtgttttgggcgtgaaaagcgagtgcgtgggaatacatggggcatgcagtggccagaccaagtgccaggccccatgccccgaaacctccctcgcggggacaagcgatcccctcacggaaccctaaaacctggttgtttaggggttaaaccccctccctgtaataccaaagtgctgc

SEQ ID NO: 10 - protein, 1087 aa, Chlamydomonas reinhardtii
MGPTPSWAARGRAAVALSASLFFQLLVLAGCATFAFFMFVTPTRWYLVREAEFIAWRNSLILPYTFINGTTGRTTPLLSGTISWLVIWQWTTTLAAALGAAGLLWVASLNSASSPTPNKSSSSKRPQACWSLGRAIKRALTRARRVLHHPLPPRGMWRTLFGPGGLSVLDMFLILFWFAIHAMWMYEMTMRVLDNRRANPPPPRPRSPPPASAARPLPAASTSSSTNASASSGTASSPRPAARLLLGATHSRTLLQSQSQSQSAYPPPPKSANSTKAAAPPPPTVRRLKPLPEVVQDSVAKYSGWVGRLDLLLLFFPLPRCNFLHWLLGSDFPTLVKYHRWLGHGTMLINSIHGIVYMGMWANAGTLTTMLNWGMGAGVNRLAGLISLCGGWLLWITCIPIIRRRFFNVFYISHILGAIIFLLFGFMHRKDVATWVMPGIFLYLLDVVLRTIQQCFNATRVSLVASPADTPASIKRAVMSMPQGGSGGVAASLSRDGGVLTLTIPCDQSMTWCGGDIVFLNLPCVSWWQWHPFTIANTSATFDHPAAATPSPGAEPHFAKDSSMSSTSASSGAAAAAASGARVRYMQVHIKKYNAWTRSLIKQLEASGGASSLALYVSGPYHPPTLTGSNSYSRRLVNGFRRHVFIAGGIGVTPALGMIQELIAARRNTNTNNNTNNTNTGTNDDKAHGRVTFVWVSRSADELEAVLPAEVLEEANRGKDGWLDMQLYITGSSNNSNSKAAAASAITVDVAADANTAAGAADASAAAAAEAKLASLTPAFSRSYATFSHALSGLSGLRRSGSNSSSGDIGSGLAGGFFGFRSVWDPVAAVVFLPGYLLVLAAGAVRWAAFGAVKARPLAHPYIAGGPLMWAVAVVLCFAGGFGGLITAQSYDGYAARTVAVRSDYSYVGMLQFAALGVGAVLGPALLALVMHAMCRLLSLKRRTANVTEMESQPSASGSDASPADCRIGVTVTAGTNAATGAASKALAAPPPAHVPTAAAYISTGRPDLKTILTHAAAPYAPDGEAVEDDQAAAVGVFVAGPAPLVAAVADTCAAVNGVWAALFGGCCGGGVGGRAYLELHALTHEL

(19) United States
(12) Patent Application Publication
(10) Pub. No.: US 2013/0210064 A1
(43) Pub. Date: Aug. 15, 2013

(54) MAGNETOTACTIC ALGAE AND METHODS OF USE

(71) Applicants: Pulak Nath, Los Alamos, NM (US); Scott N. Twary, Santa Fe, NM (US)

(72) Inventors: Pulak Nath, Los Alamos, NM (US); Scott N. Twary, Santa Fe, NM (US)

(73) Assignee: Los Alamos National Security, LLC,

(21) Appl. No.: 13/494,533

(22) Filed: Jun. 12, 2012

Related U.S. Application Data
(60) Provisional application No. 61/496,379, filed on Jun. 13, 2011.

(51) Int. Cl. C12P 7/64; C12R 1/89; C12P 3/00; C12N 15/79

(52) U.S. Cl. CPC C12P 7/6409; C12N 15/79; C12R 1/89; C12P 3/00
USPC 435/34; 435/257.2; 435/471; 435/134; 435/168; 435/6.17

(57) ABSTRACT

Disclosed herein are magnetotactic algae, such as algae cells that include magnetic nanoparticles. In some examples the magnetotactic algae express a nucleic acid molecule encoding a bacterial MagA ferrous transporter, a nucleic acid molecule encoding a bacterial Mms6 magnetite binding protein, or both. Also disclosed herein are methods for producing magnetotactic algae and methods of producing biofuel or magnetic nanoparticles utilizing magnetotactic algae. Further disclosed herein are methods of enriching a population of magnetotactic algae cells (for example, increasing the number of magnetotactic algae cells in a population of algae cells). In further embodiments, disclosed herein are methods of selecting a transformed algae cell.

### CROSS REFERENCE TO RELATED APPLICATION

This claims the benefit of U.S. Provisional Application No. 61/496,379, filed Jun. 13, 2011, which is incorporated by reference herein in its entirety.

### ACKNOWLEDGMENT OF GOVERNMENT SUPPORT

This invention was made with government support under Contract No. DE-AC52-06NA25396 awarded by the U.S. Department of Energy. The government has certain rights in the invention.

### FIELD

This disclosure relates to algae cells including magnetic nanoparticles and methods for their production and use.

### BACKGROUND

There is continuing interest in the production of fuel from algae as an alternative energy source. However, commercial production of algal biofuel as a cost-competitive replacement for fossil fuel faces several technological barriers. Two key barriers are low cost algal harvesting and oil extraction. Harvesting and oil extraction account for about 30% of the total cost of production. Current technologies available for harvesting and extraction are either too expensive or not scalable for commercial production.

### SUMMARY

Disclosed herein are magnetotactic algae, such as algae cells that include magnetic nanoparticles. In some examples the magnetotactic algae express a nucleic acid molecule encoding a bacterial MagA ferrous transporter (such as a Magnetospirillum magneticum MagA nucleic acid), a nucleic acid molecule encoding a bacterial Mms6 magnetite binding protein (such as a Magnetospirillum magneticum Mms6 nucleic acid), or both. In one example, the magnetotactic alga is a Chlamydomonas cell (such as C. reinhardtii), that expresses a bacterial MagA ferrous transporter and/or a bacterial Mms6 magnetite binding protein. In another example, the magnetotactic alga is a Nannochloropsis cell (such as Nannochloropsis salina) that expresses a bacterial MagA ferrous transporter and/or a bacterial Mms6 magnetite binding protein.

Also disclosed herein are methods for producing magnetotactic algae and methods of producing biofuel or magnetic nanoparticles utilizing magnetotactic algae. In some embodiments, the disclosed methods include methods of producing magnetotactic algae. The methods include transforming an alga cell with a nucleic acid molecule encoding a bacterial MagA ferrous transporter and/or a nucleic acid molecule encoding a bacterial Mms6 magnetite binding protein and cultivating the transformed alga cell and/or progeny thereof that express the bacterial MagA ferrous transporter, the bacterial Mms6 magnetite binding protein, or both, under conditions sufficient to produce algae cells including magnetic nanoparticles, thereby producing magnetotactic algae. In some examples, the methods further include collecting the algae including the magnetic nanoparticles, for example, magnetically collecting the algae.

Further disclosed herein are methods of enriching a population of magnetotactic algae cells (for example, increasing the number of magnetotactic algae cells in a population of algae cells). The methods include transforming an alga cell with a nucleic acid molecule encoding a bacterial MagA ferrous transporter and/or a nucleic acid molecule encoding a bacterial Mms6 magnetite binding protein, cultivating the transformed alga cell and/or progeny thereof that express the bacterial MagA ferrous transporter, the bacterial Mms6 magnetite binding protein, or both, under conditions sufficient to produce algae including magnetic nanoparticles, and magnetically collecting the algae including magnetic nanoparticles. In some examples, the magnetically collected algae are subsequently cultivated under conditions sufficient to produce algae including magnetic nanoparticles and magnetically collecting the algae cells including magnetic nanoparticles, further enriching the population of magnetotactic algae cells.

In some embodiments, disclosed herein are methods of producing biofuel. The methods include cultivating an alga cell expressing a nucleic acid molecule encoding a bacterial MagA ferrous transporter and/or a nucleic acid encoding a bacterial Mms6 magnetite binding protein, and/or progeny thereof, under conditions sufficient to produce algae including magnetic nanoparticles, magnetically collecting the algae including magnetic nanoparticles, and extracting lipid from the collected algae, thereby producing biofuel. In some examples, the methods include lysing the algae cell to extract lipid, for example by exposing the algae including magnetic nanoparticles to an alternating magnetic field.

In other embodiments, disclosed herein are methods of producing magnetic nanoparticles. The methods include cultivating an alga cell expressing a nucleic acid molecule encoding a bacterial MagA ferrous transporter and/or a nucleic acid encoding a bacterial Mms6 magnetite binding protein, and/or progeny thereof, under conditions sufficient to produce algae including magnetic nanoparticles, magnetically collecting the algae including magnetic nanoparticles, and isolating the magnetic nanoparticles from the collected algae. In some examples, the methods include lysing the algae cell to isolate the magnetic nanoparticles, for example by exposing the algae including magnetic nanoparticles to an alternating magnetic field.

In further embodiments, disclosed herein are methods of selecting a transformed algae cell. The methods include cultivating an alga cell transformed with a first nucleic acid molecule encoding a bacterial MagA ferrous transporter and/or a nucleic acid encoding a bacterial Mms6 magnetite binding protein, and/or progeny thereof that express the bacterial MagA ferrous transporter, the bacterial Mms6 magnetite binding protein, or both, under conditions sufficient to produce algae including magnetic nanoparticles, and magnetically collecting the algae comprising magnetic nanoparticles, thereby selecting the transformed algae cell. In some examples, the algae cell is further transformed with a second nucleic acid molecule encoding a protein of interest.

In some examples, the bacterial MagA ferrous transporter is a Magnetospirillum magneticum MagA ferrous transporter, for example encoded by a nucleic acid molecule including the sequence of SEQ ID NO: 1 or SEQ ID NO: 3. In additional examples, the bacterial Mms6 magnetite binding protein is a Magnetospirillum magneticum Mms6 magnetite binding protein, for example encoded by a nucleic acid molecule including the sequence set forth as SEQ ID NO: 4, SEQ ID NO: 5, or SEQ ID NO: 8. Exemplary algae of use in the disclosed compositions and methods include Chlamydomonas (such as C. reinhardtii), Nannochloropsis, Botryococcus, Chlorella, Dunaliella, Gracilaria, Pleurochrysis, or Sargassum.

The foregoing and other features of the disclosure will become more apparent from the following detailed description, which proceeds with reference to the accompanying figures.

### BRIEF DESCRIPTION OF THE DRAWINGS

FIG. 1 is a digital image showing response of C. reinhardtii transformed with MagA (left) as compared to wild-type (non-transformed) algae (right). Transformed algae were cultured in liquid media including 100 μM ferrous sulfate. The magnet is behind the tube. Wild-type algae settled to the bottom of the tube, however, transformed algae did not settle, and were also drawn to the side of the tube closest to the magnet.

FIG. 2 is a pair of digital images of micrographs obtained by transmission electron microscopy of a wild-type (untransformed) Chlamydomonas reinhardtii cell (left) and a Chlamydomonas reinhardtii cell transformed with MagA (right). Aggregates of magnetic nanoparticles (circled) are visible in the transformed cell.

### SEQUENCE LISTING

The nucleic acid and amino acid sequences listed herein are shown using standard letter abbreviations for nucleotide bases, and one letter code for amino acids.

Only one strand of each nucleic acid sequence is shown, but the complementary strand is understood as included by any reference to the displayed strand.

The Sequence Listing is submitted as an ASCII text file in the form of the file named Sequence_Listing.txt, which was created on Jun. 8, 2012, and is 27,115 bytes, which is incorporated by reference herein.

SEQ ID NOs: 1 and 2 are exemplary nucleic acid and amino acid sequences, respectively, of a Magnetospirillum magneticum AMB-1 MagA ferrous transporter.

SEQ ID NO: 3 is an exemplary nucleic acid sequence of a MagA ferrous transporter codon-optimized for Chlamydomonas. SEQ ID NOs: 4 and 5 are exemplary nucleic acid sequences of a Magnetospirillum magneticum AMB-1 Mms6 magnetite binding protein.

SEQ ID NOs: 6 and 7 are exemplary amino acid sequences of a Magnetospirillum magneticum AMB-1 Mms6 magnetite binding protein.

SEQ ID NO: 8 is an exemplary nucleic acid sequence of an Mms6 magnetite binding protein codon-optimized for Chlamydomonas.

SEQ ID NOs: 9 and 10 are exemplary nucleic acid and amino acid sequences, respectively, of a Chlamydomonas reinhardtii Fre1 ferrireductase.

### DETAILED DESCRIPTION

Disclosed herein are magnetotactic algae which express one or more nucleic acids encoding proteins involved in iron uptake, metabolism, or production of magnetic nanoparticles. The disclosed algae can be rapidly and cost-effectively separated from a solution by application of a magnetic field (for example using a rare earth magnet), providing advantages in production of biofuels, wastewater treatment, and selection of genetically engineered algae. For example, harvesting of algae by conventional methods currently accounts for up to 15-20% of the overall cost of biofuel production from algae. By utilizing the disclosed magnetotactic algae, the algae can be harvested by magnetic separation, a low-cost, low-maintenance, and highly scalable separation method, which can reduce harvesting costs by up to 90%. Similarly, the disclosed magnetotactic algae can be used for wastewater treatment (for example, removal of heavy metals) and can be cost effectively removed from the treated water by magnetic separation. Finally, production of magnetic nanoparticles can be utilized as a selectable marker for transformed algal cells, for example, in place of conventional antibiotic resistance markers. Cells can be transformed with one or more of the nucleic acids disclosed herein and co-transformed with one or more additional nucleic acids of interest. Magnetic separation can be utilized to isolate transformed cells, eliminating problems associated with the natural antibiotic resistance of some algae, potential exposure to antibiotics or antibiotic resistance genes from cells or products, and the costs of antibiotics.

The disclosed algae are also useful for the production of magnetic nanoparticles, which can be isolated from the algae cells. The magnetic nanoparticles are suitable for many applications. These include biomedical applications, such as imaging (for example as MRI contrast agents) or therapeutic use (for example, tumor therapy by magnetic hyperthermia); electronics (for example, for high-density data storage); pigments; or as environmental catalysts (for example, for oxidation of carbon monoxide or oxidative pyrolysis of biomass). Production of magnetic nanoparticles by the disclosed biological methods is advantageous because it does not require the use of toxic precursors or organic solvents. In addition, use of algae to produce the magnetic nanoparticles does not require special growth conditions or oxygen concentrations (as is required for culture of anaerobic magnetotactic bacteria).

### I. Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” All publications, patent applications, patents, and other references mentioned herein are incorporated by reference in their entirety. In case of conflict, the present specification, including explanations of terms, will control. In addition, the materials, methods, and examples are illustrative only and not intended to be limiting.

In order to facilitate review of the various embodiments of the invention, the following explanations of specific terms are provided:

Algae: A group of autotrophic organisms which range from unicellular to multicellular forms. Unicellular algae are commonly referred to as microalgae. Microalgae include Achnanthes, Amphora, Borodinella, Botryococcus, Chaetoceros, Chlorococcum, Chlorella, Chlamydomonas, Cyclotella, Dunaliella, Pleurochrysis, Monoraphidium, Nannochloris, Nannochloropsis, Navicula, Nitzschia, Oocysitis, Oscillatoria, Phaeodactylum, Stichococcus, Synechococcus, Tetraselmis, and Thalassiosira. Macroalgae (seaweed) include Gracilaria and Sargassum. The singular form of algae is alga.

Cultivation: Intentional growth of an organism or cell, such as an alga (for example, Chlamydomonas or Nannochloropsis) in the presence of assimilable sources of carbon, nitrogen and mineral salts. In an example, such growth can take place in a solid or semi-solid nutritive medium, or in a liquid medium in which the nutrients are dissolved or suspended. In a further example, the cultivation may take place on a surface or by submerged culture. The nutritive medium can be composed of complex nutrients or can be chemically defined.

Heterologous: Originating from a different genetic sources or species. A gene that is heterologous to an alga cell originates from an organism or species other than the algal cell in which it is expressed. In one specific, non-limiting example, a heterologous MagA or Mms6 gene includes a Magnetospirillum magneticum gene which is expressed in an algal cell (for example a Chlamydomonas cell). Methods for introducing a heterologous gene into an alga cell are well known in the art, for example transformation with a nucleic acid, including electroporation, lipofection, and particle gun acceleration.

Isolated: An “isolated” component (such as a magnetic nanoparticle, nucleic acid molecule, protein, or cell) has been substantially separated or purified away from other components in the cell of the organism, or the organism itself, in which the component occurs, such as other chromosomal and extra-chromosomal DNA and RNA, proteins and cells. Magnetic nanoparticles that have been “isolated” include magnetic nanoparticles purified by standard purification methods. For example, an isolated magnetic nanoparticle can be a magnetic nanoparticle that is substantially separated from other cell components.

MagA: A bacterial ferrous (Fe²⁺) transporter. Nucleic acid and protein sequences for MagA are publicly available. For example, GenBank Accession No. NC_{—}007626 (nucleotides 4400031 to 4401335; SEQ ID NO: 1) provides an exemplary Magnetospirillum magneticum AMB-1 MagA nucleic acid sequence, and GenBank Accession No. YP_{—}423353 (SEQ ID NO: 2) provides an exemplary MagA protein sequence; both of these publicly available sequences are incorporated by reference as provided in GenBank on Jun. 13, 2011. Additional MagA sequences are available, for example from additional Magnetospirillum magneticum strains (such as strain MGT-1) or from Magnetospirillum magnetotacticum (such as strain MS-1) and can be identified by one of ordinary skill in the art.

In one example, MagA includes a full-length wild-type (or native) sequence, as well as MagA allelic variants that retain ferrous transporter activity. In certain examples, MagA has at least 80% sequence identity, for example at least 85%, 90%, 95%, or 98% sequence identity to a publicly available MagA sequence, such as those disclosed herein.

Magnetic nanoparticle: A nanoparticle containing magnetite (Fe₂O₃). In some examples, a magnetic nanoparticle is produced by a living cell, such as a bacterial or algal cell. In some examples, a magnetic nanoparticle produced by a living cell is included in an organelle in a cell (for example, a magnetosome). However, formation of a magnetosome is not required for formation of magnetic nanoparticles.

Magnetotactic: Orienting in a magnetic field along the magnetic field lines. Magnetotactic algae are algae that include intracellular magnetic nanoparticles and are capable of orienting along magnetic field lines. Magnetotactic cells (such as magnetotactic algae cells) can be collected magnetically, for example, collected based on application of or exposure to a magnetic field.

Mms6: A bacterial protein tightly associated with magnetite crystals. Mms6 is believed to play a role in regulation of magnetite crystal size and morphology in magnetotactic bacteria (e.g., Tanaka et al., J. Biol. Chem. 286:6386-6392, 2011). Nucleic acid and protein sequences for Mms6 are publicly available. For example, GenBank Accession Nos. AB096081 (SEQ ID NO: 4) and NC_{—}007626 (nucleotides 1016676-1017149; SEQ ID NO: 5) provide exemplary Magnetospirillum magneticum AMB-1 Mms6 nucleic acid sequences, and GenBank Accession Nos. BAC65162 (SEQ ID NO: 6) and YP_{—}420319 (SEQ ID NO: 7) provide exemplary Mms6 protein sequences; all of these publicly available sequences are incorporated by reference as provided in GenBank on Jun. 8, 2012. Additional Mms6 sequences are available, for example from additional Magnetospirillum magneticum strains (such as strain MGT-1) or from Magnetospirillum gryphiswaldense and can be identified by one of ordinary skill in the art.

In one example, Mms6 includes a full-length wild-type (or native) sequence, as well as Mms6 allelic variants that retain magnetic particle binding activity. In certain examples, Mms6 has at least 80% sequence identity, for example at least 85%, 90%, 95%, or 98% sequence identity to a publicly available Mms6 sequence, such as those disclosed herein.

Purified: The term “purified” does not require absolute purity; rather, it is intended as a relative term. Thus, for example, a purified magnetic nanoparticle preparation is one in which the magnetic nanoparticle is more pure than the magnetic nanoparticle in its environment within a cell. For example, a preparation of magnetic nanoparticles is purified such that the magnetic nanoparticle represents at least 50% of the total content of the preparation.

Tesla (T): Tesla is the SI derived unit of magnetic field B, also known as “magnetic flux density.” One tesla is equal to one weber per square meter.

Transduced and Transformed: A virus or vector “transduces” a cell when it transfers nucleic acid into the cell. A cell is “transformed” by a nucleic acid transduced into the cell when the DNA becomes stably replicated by the cell, either by incorporation of the nucleic acid into the cellular genome, or by episomal replication. As used herein, the term transformation encompasses all techniques by which a nucleic acid molecule is introduced into such a cell, including transformation with plasmid vectors, and introduction of naked DNA by electroporation, lipofection, and particle gun acceleration.

### III. Overview of Several Embodiments

Disclosed herein are magnetotactic algae and methods of making and using such algae. In some examples the magnetotactic algae express a nucleic acid molecule encoding a bacterial MagA ferrous transporter (such as a Magnetospirillum magneticum MagA nucleic acid, for example SEQ ID NO: 1 or SEQ ID NO: 3). In other examples, the magnetotactic algae express a nucleic acid encoding a bacterial Mms6 magnetite binding protein (such as a Magnetospirillum magneticum Mms6 magnetite binding protein, for example SEQ ID NO: 4, SEQ ID NO: 5, or SEQ ID NO: 8). In still further examples, the magnetotactic algae express both a nucleic acid encoding a bacterial MagA ferrous transporter and a nucleic acid encoding a bacterial Mms6 magnetite binding protein. Without being bound by theory, it is believed that heterologous expression of a bacterial MagA ferrous transporter in algal cells facilitates iron uptake by the algae cells, permitting formation of magnetic nanoparticles, for example in magnetosomes, as compared to in the absence of a MagA expression. Further, again without being bound by theory, it is believed that heterologous expression of a bacterial Mms6 magnetite binding protein in algal cells facilitates formation of more magnetic nanoparticles, larger magnetic nanoparticles, and/or magnetic nanoparticles with more regular crystal structures than in the absence of Mms6 expression. In one example, the magnetotactic alga cell is a Chlamydomonas cell (such as C. reinhardtii), that expresses a bacterial MagA ferrous transporter, a bacterial Mms6 magnetite binding protein, or both. In another example, the magnetotactic algae are a Nannochloropsis cell (such as Nannochloropsis salina) that expresses a bacterial MagA ferrous transporter, a bacterial Mms6 magnetite binding protein, or both.

Magnetotactic algae are useful for a variety of applications, including production of biofuels and production of magnetic nanoparticles, for example for biomedical use. The disclosed magnetotactic algae can be magnetically harvested (for example, commercially available permanent magnet-based filters or rare earth magnets). The magnetotactic algae may also be lysed using magnetic hyperthermia to release lipids and/or the magnetic nanoparticles contained in the cells. The lipids can be used for biofuel production. The magnetic nanoparticles can be used for medical applications, such as MRI, cancer treatment or targeted drug delivery. In addition, biologically produced magnetic nanoparticles have superior magnetic properties compared to those produced by chemical synthesis methods. See, e.g., Prozorov et al., Adv. Funct. Mater. 17:951-957, 2007; Xie et al., Nano. Res. 2:261-278, 2009.

Also disclosed herein are methods for producing magnetotactic algae. The methods include transforming an alga cell with a nucleic acid molecule encoding a bacterial MagA ferrous transporter, a nucleic acid molecule encoding a bacterial Mms6 magnetite binding protein, or both and cultivating the transformed alga cell and/or progeny thereof that express the bacterial MagA ferrous transporter, the bacterial Mms6 magnetite binding protein, or both, under conditions sufficient to produce algae cells including magnetic nanoparticles, thereby producing magnetotactic algae. In some examples, the methods further include collecting the algae including the magnetic nanoparticles, for example, magnetically collecting the algae.

Further disclosed herein are methods of enriching a population of magnetotactic algae cells (for example, increasing the number of magnetotactic algae cells in a population of algae cells). The methods include transforming an alga cell with a nucleic acid molecule encoding a bacterial MagA ferrous transporter, a nucleic acid molecule encoding bacterial Mms6 magnetite binding protein, or both, cultivating the transformed alga cell and/or progeny thereof that express the bacterial MagA ferrous transporter, the bacterial Mms6 magnetite binding protein, or both, under conditions sufficient to produce algae including magnetic nanoparticles, and magnetically collecting the algae including magnetic nanoparticles. In some examples, the magnetically collected algae are subsequently cultivated under conditions sufficient to produce algae including magnetic nanoparticles and magnetically collecting the algae cells including magnetic nanoparticles, further enriching the population of magnetotactic algae cells.

In some embodiments, disclosed herein are methods of producing biofuel. The methods include cultivating an alga cell expressing a nucleic acid molecule encoding a bacterial MagA ferrous transporter, a nucleic acid molecule encoding a bacterial Mms6 magnetite binding protein, or both, and/or progeny thereof under conditions sufficient to produce algae including magnetic nanoparticles, magnetically collecting the algae including magnetic nanoparticles, and extracting lipid from the collected algae, thereby producing biofuel. In some examples, the methods include lysing the algae cell to extract lipid, for example by exposing the algae including magnetic nanoparticles to an alternating magnetic field.

In other embodiments, disclosed herein are methods of producing magnetic nanoparticles. The methods include cultivating an alga cell expressing a nucleic acid molecule encoding a bacterial MagA ferrous transporter, a nucleic acid molecule encoding a bacterial Mms6 magnetite binding protein, or both, and/or progeny thereof under conditions sufficient to produce algae including magnetic nanoparticles, magnetically collecting the algae including magnetic nanoparticles, and isolating the magnetic nanoparticles from the collected algae, thereby producing biofuel. In some examples, the methods include lysing the algae cell to isolate the magnetic nanoparticles, for example by exposing the algae including magnetic nanoparticles to an alternating magnetic field.

In further embodiments, disclosed herein are methods of selecting a transformed algae cell. The methods include cultivating an alga cell transformed with a first nucleic acid molecule encoding a bacterial MagA ferrous transporter, a nucleic acid molecule encoding a bacterial Mms6 magnetite binding protein, or both and/or progeny thereof that express the bacterial MagA ferrous transporter, the bacterial Mms6 magnetite binding protein, or both, under conditions sufficient to produce algae including magnetic nanoparticles, and magnetically collecting the algae comprising magnetic nanoparticles, thereby selecting the transformed algae cell. In some examples, the algae cell is further transformed with a second nucleic acid molecule encoding a protein of interest.

The disclosed methods of selecting a population of transformed cells and/or enriching a population of cells by magnetic collection are not limited to algal cells. Any cell type (for example, bacterial, fungal, plant, or mammalian cells) can be transformed with a bacterial MagA ferrous transporter and/or a bacterial Mms6 magnetite binding protein in order to produce magnetotactic cells. The methods disclosed herein with respect to algal cells can equally be applied to other cell types, with modifications for the transformation and culture of specific cell types, as is known to one of ordinary skill in the art.

### III. Expression of Bacterial MagA and/or Mms6 in Algae Cells

The disclosed magnetotactic algae and methods of making and using such algae include algae transformed with and/or expressing one or more nucleic acid molecules encoding one or more bacterial genes relating to iron transport and/or metabolism. In one embodiment, the gene encodes a bacterial MagA ferrous transporter. In another embodiment, the gene encodes a magnetite binding protein, such as bacterial Mms6. In additional embodiments, Mms6 is used in combination with MagA to produce the magnetotactic algae disclosed herein. Additional nucleic acids encoding components of the magnetosome are known to one of skill in the art (see, e.g., Xie et al., Nano. Res. 2:261-278, 2009; Prozorov et al., Adv. Funct. Mater. 17:951-957, 2007). In addition, nucleic acids encoding other iron metabolism related proteins (for example, Fre1, Fea1, and/or Fea2) could be used in combination with MagA and/or Mms6 to produce the magnetotactic algae disclosed herein. In some examples, an algal nucleic acid encoding a ferrireductase of Chlamydomonas such as Fre1 (see, e.g., Allen et al., Eukaryotic Cell 6:1841-1852, 2007) is overexpressed in the disclosed algal cells expressing MagA and/or Mms6, to reduce ferric iron in solution and facilitate uptake of ferrous iron. Fre1 nucleic acid and amino acid sequences are publicly available. GenBank Accession No. XM_{—}001692181; SEQ ID NO: 9) provides an exemplary Chlamydomonas FRE1 nucleic acid sequence, and GenBank Accession No. XP_{—}001692233 (SEQ ID NO: 10) provides an exemplary Chlamydomonas Fre1 protein sequence; both of these publicly available sequences are incorporated by reference as provided in GenBank on Jun. 8, 2012. In one example, Fre1 includes a full-length wild-type (or native) sequence, as well as Fre1 allelic variants that retain ferrireductase activity. In certain examples, Fre1 has at least 80% sequence identity, for example at least 85%, 90%, 95%, or 98% sequence identity to a publicly available Fre1 sequence, such as those disclosed herein.

In some embodiments, the disclosed algae include algae transformed with a bacterial ferrous transporter, such as a bacterial MagA nucleic acid. In some examples, the MagA ferrous transporter is a Magnetospirillum magneticum MagA ferrous transporter (for example, MagA from M. magneticum strain MS-1 or strain AMB-1). In one example, the nucleic acid molecule encoding the MagA ferrous transporter includes or consists of:

(SEQ ID NO: 1)
ATGGAACTGCATCATCCCGAACTGACCTATGCCGCCATCGTCGCCCTGGCCGCCGTGCTGTGCG
GCGGGATGATGACGCGCCTGAAGCAGCCGGCCGTCGTCGGCTACATCCTGGCGGGGGTGGTGCT
GGGACCCAGCGGCTTCGGGCTGGTGAGCAACCGCGACGCCGTGGCCACCCTGGCCGAGTTCGGC
GTGCTGATGCTGCTGTTCGTCATCGGCATGAAGCTGGACATCATCCGCTTTCTCGAAGTGTGGA
AGACGGCCATCTTCACCACGGTTCTGCAGATCGCCGGCAGCGTGGGCACGGCCCTGCTGCTGCG
TCACGGCCTGGGCTGGAGCCTGGGGCTGGCGGTGGTGCTGGGCTGTGCCGTGGCGGTGTCGTCC
ACCGCCGTAGTGATCAAGGTGCTGGAATCCTCGGACGAGCTGGACACGCCGGTCGGCCGCACCA
CCCTTGGCATCCTGATCGCCCAGGACATGGCGGTGGTGCCCATGATGCTGGTGCTGGAATCCTT
CGAGACCAAGGCGCTGCTGCCCGCCGACATGGCCCGGGTGGTGCTGTCGGTGCTGTTCCTGGTG
CTGCTGTTCTGGTGGCTGTCCAAGCGCCGCATCGACCTGCCGATCACCGCCCGGCTTTCCCGCG
ATTCTGACCTTGCCACCCTGTCGACCCTGGCCTGGTGTTTCGGCACCGCCGCCATCTCCGGCGT
GCTGGACTTGTCGCCGGCCTATGGCGCCTTCCTGGGCGGCGTGGTGCTGGGCAATTCCGCCCAG
CGCGACATGCTGTTGAAGCGTGCCCAGCCCATCGGCAGCGTGCTGCTGATGGTGTTCTTCCTGT
CCATCGGGCTGCTGCTCGACTTCAAGTTCATCTGGAAGAATCTGGGCACCGTTCTCACCCTGCT
GGCCATGGTGACCCTGTTCAAGACGGCGCTGAACGTCACGGCGCTGCGCCTGGCGCGGCAGGAC
TGGCCCAGCGCCTTCCTGGCCGGCGTGGCCCTGGCCCAGATCGGCGAGTTCTCGTTCCTGCTGG
CCGAGACCGGCAAGGCGGTCAAGCTGATCAGCGCCCAGGAGACCAAGCTGGTGGTGGCGGTCAC
CGTGCTGTCCCTGGTGCTGTCGCCGTTCTGGCTGTTCACCATGCGGCGCATGCACCGGGTGGCG
GCGGTGCATGTCCATTCGTTCCGCGATCTGGTCACGCGGCTGTATGGCGACGAGGCCCGCGCTT
TCGCCCGCACCGCGCGGCGGGCCCGTGTGCTGGTGCGGCGTGGTTCCTGGAGGGATGACCCCAA
TGCCGGACCTGGCTCTGGAATTTGA

In some embodiments, a MagA encoding nucleic acid of use in the methods disclosed herein has a nucleic acid sequence at least 70%, 75%, 85%, 90%, 95%, 96%, 97%, 98% or 99% identical to the nucleic acid sequence set forth in SEQ ID NO: 1. Nucleic acid sequences that do not show a high degree of identity may nevertheless encode similar amino acid sequences, due to the degeneracy of the genetic code. It is understood that changes in nucleic acid sequence can be made using this degeneracy to produce multiple nucleic acid sequences that each encode substantially the same protein.

In some examples, the MagA nucleic acid molecule encodes a protein that includes or consists of the amino acid sequence set forth as:

(SEQ ID NO: 2)
MELHHPELTYAAIVALAAVLCGGMMTRLKQPAVVGYILAGVVLGPSGFGLVSNRDAVATLAEFG
VLMLLFVIGMKLDIIRFLEVWKTAIFTTVLQIAGSVGTALLLRHGLGWSLGLAVVLGCAVAVSS
TAVVIKVLESSDELDTPVGRTTLGILIAQDMAVVPMMLVLESFETKALLPADMARVVLSVLFLV
LLFWWLSKRRIDLPITARLSRDSDLATLSTLAWCFGTAAISGVLDLSPAYGAFLGGVVLGNSAQ
RDMLLKRAQPIGSVLLMVFFLSIGLLLDFKFIWKNLGTVLTLLAMVTLFKTALNVTALRLARQD
WPSAFLAGVALAQIGEFSFLLAETGKAVKLISAQETKLVVAVTVLSLVLSPFWLFTMRRMHRVA
AVHVHSFRDLVTRLYGDEARAFARTARRARVLVRRGSWRDDPNAGPGSGI

In some embodiments, the MagA polypeptide has an amino acid sequence at least 80%, 85%, 90%, 95%, 96%, 97%, 98% or 99% identical to the amino acid sequence set forth in SEQ ID NO: 2.

In some examples, the heterologous gene is codon-optimized for the cell in which it is to be expressed. Codon usage bias, the use of synonymous codons at unequal frequencies, is ubiquitous among genetic systems (Ikemura, J. Mol. Biol. 146:1-21, 1981; Ikemura, J. Mol. Biol. 158:573-97, 1982). The strength and direction of codon usage bias is related to genomic G+C content and the relative abundance of different isoaccepting tRNAs (Akashi, Curr. Opin. Genet. Dev. 11:660-666, 2001; Duret, Curr. Opin. Genet. Dev. 12:640-9, 2002; Osawa et al., Microbiol. Rev. 56:229-264, 1992). Codon usage can affect the efficiency of gene expression. Codon-optimization refers to replacement of a codon in a nucleic acid sequence with a synonymous codon (one that codes for the same amino acid) more frequently used (preferred) in the organism. Each organism has a particular codon usage bias for each amino acid, which can be determined from publicly available codon usage tables (for example see Nakamura et al., Nucleic Acids Res. 28:292, 2000 and references cited therein). For example, a codon usage database is available on the World Wide Web at kazusa.or.jp/codon. One of skill in the art can modify a nucleic acid encoding a particular amino acid sequence, such that it encodes the same amino acid sequence, while being optimized for expression in a particular cell type (such as an algae cell).

In some examples, the methods disclosed herein include a MagA ferrous transporter nucleic acid that has been codon-optimized for algae (such as Chlamydomonas or Nannochloropsis). In one example, a codon-optimized MagA nucleic acid (codon-optimized for Chlamydomonas) includes or consists of the following sequence:

(SEQ ID NO. 3)
CTATCTAGAGGATCCCCGGGTGGTCTCTTGACCatggagctgcaccaccccgagctgacctacg
ccgccatcgtggccctggccgccgtgctgtgcggcggcatgatgacccgcctgaagcagcccgc
cgtggtgggctacatcctggccggcgtggtgctgggccccagcggcttcggcctggtgagcaac
cgcgacgccgtggccaccctggccgagttcggcgtgctgatgctgctgttcgtgatcggcatga
agctggacatcatccgcttcctggaggtgtggaagaccgccatcttcaccaccgtgctgcagat
cgccggcagcgtgggcaccgccctgctgctgcgccacggcctgggctggagcctgggcctggcc
gtggtgctgggctgcgccgtggccgtgagcagcaccgccgtggtgatcaaggtgctggagagca
gcgacgagctggacacccccgtgggccgcaccaccctgggcatcctgatcgcccaggacatggc
cgtggtgcccatgatgctggtgctggagagcttcgagaccaaggccctgctgcccgccgacatg
gcccgcgtggtgctgagcgtgctgttcctggtgctgctgttctggtggctgagcaagcgccgca
tcgacctgcccatcaccgcccgcctgagccgcgacagcgacctggccaccctgagcaccctggc
ctggtgcttcggcaccgccgccatcagcggcgtgctggacctgagccccgcctacggcgccttc
ctgggcggcgtggtgctgggcaacagcgcccagcgcgacatgctgctgaagcgcgcccagccca
tcggcagcgtgctgctgatggtgttcttcctgagcatcggcctgctgctggacttcaagttcat
ctggaagaacctgggcaccgtgctgaccctgctggcgatggtgaccctgttcaagaccgccctg
aacgtgaccgccctgcgcctggcccgccaggactggcccagcgccttcctggccggcgtggccc
tggcccagatcggcgagttcagcttcctgctggccgagaccggcaaggccgtgaagctgatcag
cgcccaggagaccaagctggtggtggccgtgaccgtgctgagcctggtgctgagccccttctgg
ctgttcaccatgcgccgcatgcaccgcgtggccgccgtgcacgtgcacagcttccgcgacctgg
tgacccgcctgtacggcgacgaggcccgcgccttcgcccgcaccgcccgccgcgcccgcgtgct
ggtgcgccgcggcagctggcgcgacgaccccaacgccggccccggcagcggcatcTAAGCTAGC
GTGTGAATTGGTGACCCGAGCTCGAATTTC.

In some examples, a MagA nucleic acid molecule optionally includes one or more additional sequences, such as one or more linkers (for example, including one or more restriction enzyme sites) for to allow for insertion or deletion of the nucleic acid in a plasmid vector, for example. In one example, the MagA nucleic acid includes linkers, such as those shown in SEQ ID NO: 3 above in capital letters; however, the linkers are not required.

In additional embodiments, the disclosed algae include algae transformed with bacterial Mms6. In some examples, the Mms6 magnetite binding protein is a Magnetospirillum magneticum Mms6 magnetite binding protein (for example, Mms from M. magneticum strain MS-1 or strain AMB-1). In one example, the nucleic acid molecule encoding the Mms6 magnetite binding protein includes or consists of one of the following:

(SEQ ID NO: 4)
ATGGGCGAGATGGAGCGCGAGGGCGCCGCCGCCAAGGCCGGGGCTGCCAAGACGGGCGCCGCCA
AGACCGGAACCGTCGCCAAGACCGGCATCGCCGCCAAGACGGGTGTTGCCACCGCCGTTGCCGC
TCCGGCGGCTCCTGCCAATGTTGCCGCCGCCCAGGGCGCCGGGACCAAGGTCGCCCTTGGCGCG
GGCAAGGCCGCCGCCGGTGCCAAGGTCGTCGGTGGAACCATCTGGACCGGTAAGGGGCTGGGCC
TCGGTCTGGGTCTCGGTCTGGGCGCGTGGGGGCCGATCATTCTCGGCGTTGTTGGCGCCGGGGC
GGTTTACGCRTATATGAAGAGCCGTGATATCGAATCGGCGCAGAGCGACGAGGAAGTCGAACTG
CGCGACGCGCTGGCCTGA
(SEQ ID NO: 5)
GTGCCAGCTCAGATCGCCAACGGAGTTATTTGCCCCCCAGGGGCCCCGGCCGGAACCAAGGCCG
CCGCCGCCATGGGCGAGATGGAGCGCGAGGGCGCCGCCGCCAAGGCCGGGGCTGCCAAGACGGG
CGCCGCCAAGACCGGAACCGTCGCCAAGACCGGCATCGCCGCCAAGACGGGTGTTGCCACCGCC
GTTGCCGCTCCGGCGGCTCCTGCCAATGTTGCCGCCGCCCAGGGCGCCGGGACCAAGGTCGCCC
TTGGCGCGGGCAAGGCCGCCGCCGGTGCCAAGGTCGTCGGTGGAACCATCTGGACCGGTAAGGG
GCTGGGCCTCGGTCTGGGTCTCGGTCTGGGCGCGTGGGGGCCGATCATTCTCGGCGTTGTTGGC
GCCGGGGCGGTTTACGCGTATATGAAGAGCCGTGATATCGAATCGGCGCAGAGCGACGAGGAAG
TCGAACTGCGCGACGCGCTGGCCTGA

In some embodiments, a Mms6 encoding nucleic acid of use in the methods disclosed herein has a nucleic acid sequence at least 70%, 75%, 85%, 90%, 95%, 96%, 97%, 98% or 99% identical to the nucleic acid sequence set forth in SEQ ID NO: 4 or SEQ ID NO: 5. Nucleic acid sequences that do not show a high degree of identity may nevertheless encode similar amino acid sequences, due to the degeneracy of the genetic code. It is understood that changes in nucleic acid sequence can be made using this degeneracy to produce multiple nucleic acid sequences that each encode substantially the same protein.

In some examples, the Mms6 nucleic acid molecule encodes a protein that includes or consists of the amino acid sequence set forth as one of the following:

(SEQ ID NO: 6)
MGEMEREGAAAKAGAAKTGAAKTGTVAKTGIAAKTGVATAVAAPAAPANVAAAQGAGTKVALGA
GKAAAGAKVVGGTIWTGKGLGLGLGLGLGAWGPIILGVVGAGAVYAYMKSRDIESAQSDEEVEL
RDALA
(SEQ ID NO: 7)
MPAQIANGVICPPGAPAGTKAAAAMGEMEREGAAAKAGAAKTGAAKTGTVAKTGIAAKTGVATA
VAAPAAPANVAAAQGAGTKVALGAGKAAAGAKVVGGTIWTGKGLGLGLGLGLGAWGPIILGVVG
AGAVYAYMKSRDIESAQSDEEVELRDALA

In some embodiments, the Mms6 polypeptide has an amino acid sequence at least 80%, 85%, 90%, 95%, 96%, 97%, 98% or 99% identical to the amino acid sequence set forth in SEQ ID NO: 6 or SEQ ID NO: 7.

In some examples, the heterologous gene is codon-optimized for the cell in which it is to be expressed, as discussed above with respect to MagA. One of skill in the art can modify a nucleic acid encoding a particular amino acid sequence, such that it encodes the same amino acid sequence, while being optimized for expression in a particular cell type (such as an algae cell). In some examples, the methods disclosed herein include a Mms6 nucleic acid that has been codon-optimized for algae (such as Chlamydomonas or Nannochloropsis). In one example, a codon-optimized MagA nucleic acid (codon-optimized for Chlamydomonas) includes or consists of the following sequence:

(SEQ ID NO: 8)
ATGGGCGAGATGGAGCGCGAGGGCGCCGCCGCCAAGGCCGGCGCCGCCAAGACCGGCGCCGCCA
AGACCGGCACCGTGGCCAAGACCGGCATCGCCGCCAAGACCGGCGTGGCCACCGCCGTGGCCGC
CCCCGCCGCCCCCGCCAACGTGGCCGCCGCCCAGGGCGCCGGCACCAAGGTGGCCCTGGGCGCC
GGCAAGGCCGCCGCCGGCGCCAAGGTGGTGGGCGGCACCATCTGGACCGGCAAGGGCCTGGGCC
TGGGCCTGGGCCTGGGCCTGGGCGCCTGGGGCCCCATCATCCTGGGCGTGGTGGGCGCCGGCGC
CGTGTACGCCTACATGAAGAGCCGCGACATCGAGAGCGCCCAGAGCGACGAGGAGGTGGAGCTG
CGCGACGCCCTGGCC

In further embodiments, the disclosed algae include algae transformed with a bacterial ferrous transporter and a bacterial magnetite binding protein. In some examples, the algae are transformed with a nucleic acid encoding a Magnetospirillum magneticum MagA ferrous transporter (such as SEQ ID NO: 1 or SEQ ID NO: 3) and a nucleic acid encoding a Magnetospirillum magneticum Mms6 magnetic particle binding protein (such as SEQ ID NO: 4, SEQ ID NO: 5, or SEQ ID NO: 8). In other examples, algae are transformed with a nucleic acid at least 70% identical (for example, at least 75%, 80%, 85%, 90%, 95%, 96%, 97%, 98% or 99% identical) to the nucleic acid sequence set forth as SEQ ID NO: 1 or SEQ ID NO:3 and a nucleic acid at least 70% identical (for example, at least 75%, 80%, 85%, 90%, 95%, 96%, 97%, 98% or 99% identical) to the nucleic acid sequence set forth as SEQ ID NO: 4, SEQ ID NO: 5, or SEQ ID NO: 8. In additional embodiments, the algae are further transformed with an algal Fre1 ferrireductase nucleic acid, such as SEQ ID NO: 9, or a nucleic acid at least 70% identical (for example, at least 75%, 80%, 85%, 90%, 95%, 96%, 97%, 98% or 99% identical) to SEQ ID NO: 10.

Methods of transforming algae are known in the art. In an embodiment, a nucleic acid molecule can be inserted into one or more expression vectors, using methods known to those of skill in the art. Vectors include one or more expression cassettes including expression control sequences operably linked to the nucleic acid of interest (such as MagA, Mms6, Fre1, or other nucleic acids). An expression cassette includes nucleic acid elements that permit expression of a gene in a host cell. Typically, the expression cassette includes a promoter that is functional in the selected host system that is operably linked to the gene to be expressed. The promoter can be constitutive or inducible. In an embodiment, the expression cassette includes a promoter, ribosome binding site, a start codon (ATG) if necessary, and optionally a region encoding a leader peptide in addition to the desired DNA molecule and stop codon. In addition, a 3′ terminal region (translation and/or transcription terminator) can be included within the cassette. The heterologous nucleic acid constituted in the DNA molecule may be solely controlled by the promoter so that transcription and translation occur in the host cell. In one example, the nucleic acid is placed under the control of the strong constitutive PSAD promoter. Additional constitutive promoters suitable for use in algae cell transformation include the ribulose bisphosphate carboxylase (RBCS2) promoter or the HSP70A-RBCS2 tandem promoter. Inducible promoters of use in algae cell transformation include NIT1 (ammonium starvation-induced), CA1 (low CO₂-induced), and CYC6 (induced by copper depletion or nickel or cobalt addition) promoters.

The presence of additional regulatory sequences within the expression cassette may be desirable to allow for regulation of expression of the nucleic acid relative to the growth of the host cell. These regulatory sequences are well known in the art. Examples of regulatory sequences include sequences that turn gene expression on or off in response to chemical or physical stimulus as well as enhancer sequences. In addition, to the regulatory sequences, selectable markers can be included to assist in selection of transformed cells. For example, genes that confer antibiotic resistance or sensitivity to the plasmid may be used as selectable markers.

Transformation of an alga cell with recombinant DNA can be carried out by conventional techniques as are well known to those of ordinary skill in the art. Methods of transformation include transformation utilizing Agrobacterium tumifaciens transformed with a plasmid including the desired nucleic acid, such as MagA, Mms6, and/or FRE1 (for example, a pCAMBIA vector). Other methods include biolistics (e.g., a “gene gun”), for example utilizing a pSL18 plasmid including the nucleic acid. In other examples, algae cells can be transformed utilizing electroporation, glass beads, or carbide whiskers. One of ordinary skill in the art can select an appropriate transformation method and vector, based on the cells to be transformed and other desired characteristics.

A wide variety of algae species (such as microalgae and/or macroalgae) can be utilized in the methods described herein. In some examples, the algae species include, but are not limited to Chlorella (such as Chlorella vulgaris), Chlamydomonas (such as Chlamydomonas reinhardtii), Chaetoceros, Spirulina (such as Spirulina platensis), Dunaliella, and Porphyridum. In particular examples, the algae species include algae useful for production of biofuels or other compounds (such as polyunsaturated acids, pigments, or phytochemicals, for example, for nutritional supplements). In some examples, the algae include Akistrodesmus, Arthrospira, Botryococcus braunii, Chlorella (such as Chlorella sp. or Chlorella protothecoides), Crypthecodinium (such as Crypthecodinium cohnii), Cyclotella, Dunaliella tertiolecta, Gracilaria, Hantzschia, Haematococcus (such as Haematococcus Nannochloris, Nannochloropsis, Neochloris oleoabundans, Nitzschia, Phaeodactylum, Pleurochrysis carterae (also called CCMP647), Porphyridium, Sargassum, Scenedesmus (such as Scenedesmus obliquus), Schiochytrium, Stichococcus, Tetraselmis, Thalassiosira pseudonana, Thraustochytrium roseum, and Ulkenia sp. In one example, the algae species is Chlamydomonas reinhardtii. In another example, the algae cells are Nannochloropsis salina. In another example, the algae cells are Tetraselmis striata.

### IV. Cultivation, Collection, and Lysis of Magnetotactic Algae

The algae cells disclosed herein which have been transformed with a nucleic acid molecule encoding a bacterial MagA ferrous transporter and/or a bacterial Mms6 magnetite binding protein, or which express a nucleic acid encoding a bacterial MagA ferrous transporter and/or a bacterial Mms6 magnetite binding protein are cultivated under conditions sufficient to produce algae including magnetic nanoparticles.

Methods of cultivating algae are well known to one of ordinary skill in the art. The magnetotactic algae of the present disclosure can be cultured in conventional fermentation bioreactors, which include, but are not limited to, batch, fed-batch, cell recycle, and continuous fermenters. The cultivation can also be conducted in shake flasks, test tubes, microtiter dishes, or petri plates. The algae can also be cultivated in outdoor open ponds. Cultivation of the algae is carried out at a temperature, pH and oxygen content appropriate for the particular recombinant algae species. Such culturing conditions are well within the expertise of one of ordinary skill in the art.

In some examples, the algae are cultivated in a liquid medium with shaking, such as tris-acetate phosphate medium. The algae can be grown on a standard light/dark regimen, for example about 16 hours light and 8 hours dark alternating. In some examples, the liquid medium is supplemented with Fe²⁺ (such as ferrous citrate, ferrous sulfate, or ferrous chloride) in an amount sufficient for the algae to take up iron and form magnetic nanoparticles. In some examples, the liquid medium includes about 10 μM to about 500 μM ferrous salt (such as about 20 μM to about 250 μM, about 50 μM to about 200 μM, or about 100 μM to 200 μM). In other examples, the liquid medium is not supplemented with iron, for example if the algae are transformed with or overexpress an algal Fre1 ferrireductase, which in some examples permits reduction of ferric iron naturally present in the water source to be reduced to the ferrous form and thus available for uptake by the algae cells. In one non-limiting example, the algae expressing a nucleic acid encoding a bacterial MagA ferrous transporter are cultured in tris-acetate phosphate media supplemented with 100 μM ferrous sulfate. In some examples, the algae are cultured in the liquid medium for about 12 hours or more, for example, about 12, hours, 24 hours, 36 hours, 48 hours, 60 hours, 72 hours, 84 hours, 96 hours, or more. In further examples, the algae are cultured for about 1 day to about 10 days, such as about 1, 2, 3, 4, 5, 6, 7, 8, 9, or 10 days. In one example, the algae are cultured for at least 24 hours.

Algae containing magnetic particles, such as those cultured in conditions described above, can be magnetically collected due to the presence of magnetic nanoparticles in the algae cells. Magnetic collection or separation can be carried out in a continuous flow, allowing processing of large volumes. In some examples, a high gradient magnetic sorter is used. In other examples, an open gradient magnetic sorter is used. The use of permanent magnets can substantially reduce energy consumption of the process, as compared to electromagnets. In some examples, a magnetic sorter is utilized to collect the algae which contain magnetic nanoparticles. In some examples, the algae are collected with a magnetic field of about 0.3 to 2.0 T (for example, about 0.5 to about 1.0 T, about 1.0 to about 2.0 T, for example about 2.0 T with a linear gradient). Commercial permanent magnet-based systems are available and are capable of processing up to 1000 liters/minute (e.g., magnetic filters available from Eclipse Magnetics, Sheffield, UK). However, any form of magnetic collection or separation can be utilized.

In some embodiments, the methods include lysing the algae cells to extract lipid (for example for biofuel production) or to isolate the magnetic nanoparticles (for example for use in medical, electronic, or chemical applications). Methods of lysing algae cells are known to one of skill in the art and include sonication or mechanical disruption (for example using a French press or glass beads). In the disclosed methods, algae cells including magnetic nanoparticles can also be lysed by exposing the cells to an alternating magnetic field, thereby creating localized heating (magnetic hyperthermia). Methods of producing magnetic hyperthermia in single-cell organisms (such as bacteria) are known to one of skill in the art (e.g., Timko et al., J. Magnetism Magnetic Mater. 321:1521-1524, 2009). In some examples, magnetic hyperthermia is produced utilizing a system including a sine-wave power oscillator and an induction coil, which is placed around a vessel containing the algae sample. In one example, magnetic hyperthermia is produced at a frequency of 750 kHz vs. AC-field amplitude of 0 to about 5 kA/m (such as about 0.25, 0.5, 0.75, 1.0, 1.25, 1.5, 1.75, 2.0, 2.5, 3.0, 3.5, 4.0, 4.5, or 5.0 kA/m).

Methods of extracting lipid from cells (such as algae) are well known to one of ordinary skill in the art. Suitable methods include, but are not limited to hexane solvent extraction, Soxhlet extraction, supercritical fluid extraction, extraction/expeller press, and ultrasonic-assisted extraction. See, e.g., Brennan and Owende, Renewable and Sustainable Energy Reviews 14:557-577, 2010.

In some embodiments, the methods further include isolating magnetic nanoparticles from the lysed algae cells. In some examples, the cells are disrupted by physical or chemical means. A magnetic sorter can then be utilized to sort or collect the magnetic particles from the lysed samples. Conventional methods of collecting magnetic nanoparticles can be used. Magnetic separators are commercially available, for example, from Dynal (Invitrogen, Carlsbad, Calif.), Polysciences (Warrington, Pa.), and Sigris Research (Brea, Calif.).

The following examples are provided to illustrate certain particular features and/or embodiments. These examples should not be construed to limit the invention to the particular features or embodiments described.

### EXAMPLES

### Example 1

### Production of Magnetotactic Algae

Chlamydomonas reinhardtii strain 1690 was transformed by a biolistic method with pSL18 plasmid including a nucleic acid molecule encoding MagA bacterial ferrous transporter (SEQ ID NO: 3). Transformants were selected using paramomycin. The transformed cells were cultured in tris-acetate phosphate (TAP) medium, and in TAP supplemented with 100 μM Fe²⁺. Liquid cultures were grown under 16 hour light/8 hour dark regimen, shaken at 120 rpm. A range of iron concentrations (10 μM to 200 μM) and iron salts were tested.

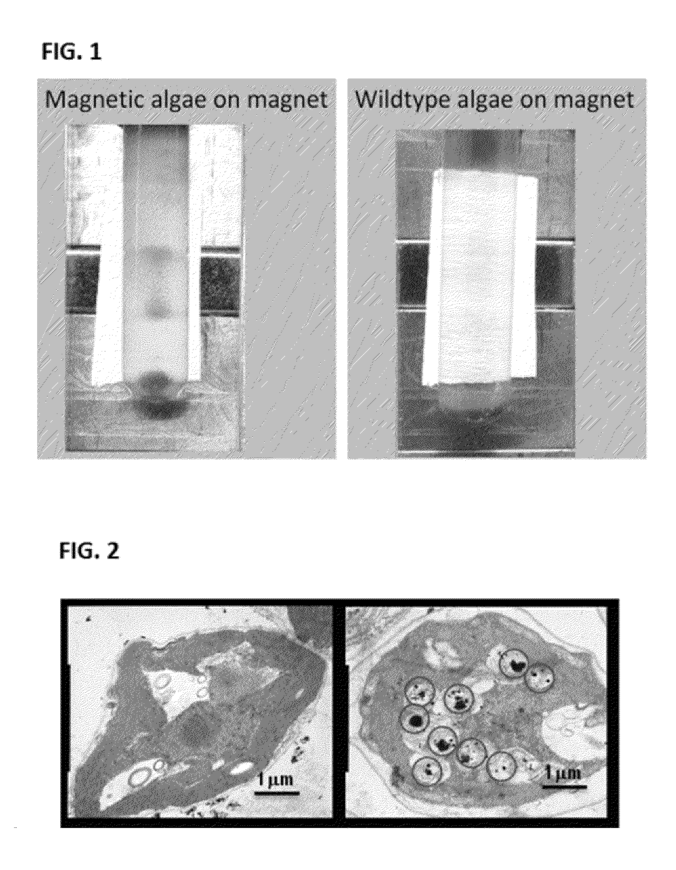

Production of magnetic nanoparticles by the transformed cells was tested by placing the culture next to a magnet and observing whether the cells were attracted to the magnet. In general, cells cultured with 10 μM Fe²⁺ did not respond to the external magnet, cells cultured with 25 μM Fe²⁺ showed a weak response, cells cultured with 50 μM Fe²⁺ showed a moderate effect, and cells cultured with 100 μM or 200 μM Fe²⁺ showed similar greatest effects. An example of the response of transformed Chlamydomonas cells grown in liquid medium including 100 μM ferrous sulfate to the external magnet is shown in FIG. 1. Similar results were obtained with transformed Nannochloropsis and Tetraselmis cells.

Transmission electron microscopy was used to visualize accumulation of magnetic nanoparticles in the transformed Chlamydomonas cells. The transformed cells included aggregates of nanoparticles within the cells (FIG. 2). The nanoparticles were estimated to have a diameter of about 10 nm.

### Example 2

### Magnetic Collection of Magnetotactic Algae

Algae including magnetic nanoparticles (such as those described in Example 1) are magnetically collected. The liquid culture containing the magnetotactic algae is passed through a high gradient magnetic sorter (HGMS) in a continuous flow. The algae are exposed to a magnetic field gradient. Algae containing magnetic nanoparticles are retained in the HGMS and the liquid medium and other non-magnetic components pass through in the flow-through.

Alternatively, algae including magnetic nanoparticles (such as those described in Example 1) are magnetically collected. The liquid culture containing the magnetotactic algae is passed through an open gradient magnetic sorter (OGMS) in a continuous flow. The algae are exposed to a magnetic field gradient. Algae containing magnetic nanoparticles are deflected in the flow and separated from the liquid medium and other non-magnetic components.

### Example 3

### Cell Lysis by Magnetic Hyperthermia

Algae including magnetic nanoparticles (such as those described in Example 1) are lysed by magnetic hyperthermia. The liquid culture containing the magnetotactic algae, or the collected magnetotactic algae (for example, collected as described in Example 2) are placed in a system including a sine-wave power oscillator and a vessel containing the algae, which is wound in an induction coil. The sample is heated by exposing the algae to a frequency of 750 kHz versus an AC-field amplitude of about 2.5 kA/m. The heating of the algae from the alternating magnetic field results in lysis of the cells. Lipid or magnetic nanoparticles or both can then be isolated from the lysed cells. Magnetic particles are collected using a magnetic sorter. Lipids are collected by standard methods in the art, such as organic extraction.

In view of the many possible embodiments to which the principles of the disclosure may be applied, it should be recognized that the illustrated embodiments are only examples and should not be taken as limiting the scope of the invention. Rather, the scope of the invention is defined by the following claims. We therefore claim as our invention all that comes within the scope and spirit of these claims.

We claim:

1. A magnetotactic alga cell, comprising an alga cell expressing a nucleic acid encoding a bacterial MagA ferrous transporter, a nucleic acid encoding a bacterial Mms6 magnetite binding protein, or a combination thereof.

2. The magnetotactic alga cell of claim 1, wherein the nucleic acid encoding the bacterial MagA ferrous transporter or the nucleic acid encoding the bacterial Mms6 magnetite binding protein is from Magnetospirillum magneticum.

3. The magnetotactic alga cell of claim 1, wherein the algal cell is Chlamydomonas, Nannochloropsis, Tetraselmis, Botryococcus, Chlorella, Dunaliella, Gracilaria, Pleurochrysis, or Sargassum.

4. A method of producing magnetotactic algae, comprising:
transforming an alga cell with a nucleic acid encoding a bacterial MagA ferrous transporter, a nucleic acid encoding a bacterial Mms6 magnetite binding protein, or a combination thereof; and
cultivating the transformed alga cell or progeny thereof that express the bacterial MagA ferrous transporter, the bacterial Mms6 magnetite binding protein, or both, under conditions sufficient to produce algae comprising magnetic nanoparticles, thereby producing magnetotactic algae.

5. The method of claim 4, further comprising magnetically collecting the algae comprising magnetic nanoparticles.

6. The method of claim 5, wherein magnetically collecting the algae comprising magnetic nanoparticles comprises enriching a population of magnetotactic algae cells.

7. The method of claim 4, wherein the nucleic acid encoding the bacterial MagA ferrous transporter or the nucleic acid encoding the bacterial Mms6 magnetite binding protein is from Magnetospirillum magneticum.

8. The method of claim 4, wherein the algal cell is Chlamydomonas, Nannochloropsis, Tetraselmis, Botryococcus, Chlorella, Dunaliella, Gracilaria, Pleurochrysis, or Sargassum.

9. The method of claim 4, wherein the conditions sufficient to produce algae comprising magnetic nanoparticles comprise a culture medium comprising at least about 25 μM Fe²⁺ salt.

10. A method of producing a product from algal cells, comprising:
cultivating an alga cell expressing a nucleic acid encoding a bacterial MagA ferrous transporter, a nucleic acid encoding a bacterial Mms 6 magnetite binding protein, or a combination thereof, or progeny thereof that express the bacterial MagA ferrous transporter, the bacterial Mms6 magnetite binding protein, or both, under conditions sufficient to produce algae comprising magnetic nanoparticles;
magnetically collecting the algae cells comprising magnetic nanoparticles; and
isolating the product from the collected algae cells.

11. The method of claim 10, wherein the product comprises a lipid, and isolating the product from the collected algae cells comprises extracting lipid from the collected algae cells.

12. The method of claim 10, wherein the product comprises magnetic nanoparticles, and isolating the product from the collected algae cells comprises lysing the algae cells.

13. The method of claim 12, wherein lysing the algae comprises exposing the algae to an alternating magnetic field.

14. The method of claim 10, wherein the nucleic acid encoding the bacterial MagA ferrous transporter or the nucleic acid encoding the bacterial Mms6 magnetite binding protein is from Magnetospirillum magneticum.

15. The method of claim 10, wherein the algal cell is Chlamydomonas, Nannochloropsis, Tetraselmis, Botryococcus, Chlorella, Dunaliella, Gracilaria, Pleurochrysis, or Sargassum.

16. The method of claim 10, wherein the conditions sufficient to produce algae comprising magnetic nanoparticles comprise a culture medium comprising at least about 25 μM Fe²⁺ salt.

17. A method of selecting a transformed algal cell, comprising:
cultivating a population of algae cells transformed with:
a first nucleic acid molecule encoding a bacterial MagA ferrous transporter, a bacterial Mms 6 magnetite binding protein, or a combination thereof, and
a second nucleic acid encoding a protein of interest,
or progeny thereof that express the bacterial MagA ferrous transporter, the bacterial Mms6 magnetite binding protein, or both, under conditions sufficient to produce algae comprising magnetic nanoparticles; and
magnetically collecting algae cells comprising magnetic nanoparticles, thereby selecting the transformed alga cell.

18. The method of claim 17, wherein the nucleic acid encoding the bacterial MagA ferrous transporter or the nucleic acid encoding the bacterial Mms6 magnetite binding protein is from Magnetospirillum magneticum.

19. The method of claim 17, wherein the algal cell is Chlamydomonas, Nannochloropsis, Tetraselmis, Botryococcus, Chlorella, Dunaliella, Gracilaria, Pleurochrysis, or Sargassum.

20. The method of claim 17, wherein the conditions sufficient to produce algae comprising magnetic nanoparticles comprise a culture medium comprising at least about 25 μM Fe²⁺ salt.
